(19) United States
(12) Patent Application Publication
(10) Pub. No.: US 2007/0134731 A1
(43) Pub. Date: Jun. 14, 2007

(54) PROTEOMICS BASED METHOD FOR TOXICOLOGY TESTING

(75) Inventor: Thomas Brown, New Baltimore, MI (US)

Correspondence Address: GIFFORD, KRASS, SPRINKLE,ANDERSON & CITKOWSKI, P.C, PO BOX 7021, TROY, MI, 48007-7021, US

(73) Assignee: Oxford Biomedical Research, Inc., Oxford, MI (US)

(21) Appl. No.: 11/674,746

(22) Filed: Feb. 14, 2007

Related U.S. Application Data
(63) Continuation of application No. 10/341,026, filed on Jan. 13, 2003.
(60) Provisional application No. 60/348,685, filed on Jan. 14, 2002.

(51) Int. Cl. G01N 33/53

(52) U.S. Cl. 435/7.1

(57) ABSTRACT

A toxicoproteomic method for developing immunoassays to determine if a toxicant or a disease causes damage to a specific organ is disclosed. The discovery phase of the method involves the administration of a toxicant to an animal which will cause damage to a specific organ of the animal, which damage results in the production of an oxidatively modified, organ specific protein(s). Blood serum, which includes the modified protein(s) released from the organ following damage, is collected from the animal. The modified protein(s) is isolated from serum by immunoaffinity purification using antibodies directed against any number of damage-related, amino acid modifications, in this instance, nitrotyrosine, chlorotyrosine and methionine sulfoxide. Its function as a biomarker is validated by determining if the modified protein(s) is specific to the tissue or organ of interest. If the modified protein is organ specific, an antibody is raised against it, and this antibody is utilized in immunoassays to (1) measure the concentration of the protein in serum and, (2) together with antibodies against the modified amino acids found on its surface, used to determine the type and extent of modification found on the protein. The assays of the present invention have utility for the toxicological screening of newly developed drugs, evaluating patients diagnostically or for organ involvement in disease, the study of cellular responses to various toxicants, and the determination of peripheral redox status.

### CROSS-REFERENCE TO RELATED APPLICATIONS

This Application is a Continuation of U.S. application Ser. 10/341,026 filed on Jan. 13, 2003 which is based on U.S. Provisional Application 60/348,685 filed on Jan. 14, 2002.

### FIELD OF THE INVENTION

This invention relates to methods for toxicological testing. More specifically, the present invention is directed to a proteomics based method of toxicological testing to identify acute and chronic organ and tissue damage.

### BACKGROUND OF THE INVENTION

Current efforts to develop new drugs, treatments and therapies for disease conditions have generated a large number of potential therapeutic agents. A number of these agents may provide the basis for effective therapies for various disease conditions; however, regulatory and ethical standards mandate that prior to human use, therapeutic agents be screened for safety. Such screening generally involves toxicological testing in a number of animal models, and with regard to various organ systems. Such testing is very expensive and time consuming, and also can raise ethical issues. Heretofore, animal toxicology studies have presented a major bottleneck in the development and implementation of new drug therapies. There is clearly a need for methods which can minimize or eliminate the burden of such animal model toxicology studies.

As will be explained in greater detail hereinbelow, the present invention is directed to a toxicoproteomic method (use of proteomic methodology to study toxicology) for evaluating tissue damage in specific organs. The method of the present invention allows for the identification of biomarkers which are specific to toxic tissue damage to particular organ systems. The method of the present invention may be employed to develop bioassays which are capable of indicating quantitative and/or qualitative damage to specific organs by compounds of interest. The methods of the present invention are based upon a method to identify circulating, organ specific modified proteins which are resultant from toxic tissue damage. These modified proteins can function as organ specific biomarkers which are indicative of tissue-specific damage. In addition to being employed for toxicological screening of compounds, the methods of the present invention may also be adapted for the development of assays which can be employed to screen a population for occupational or other environmentally based toxic insults, as well as for various disease conditions.

The methods of the present invention may be used as a substitute for animal based testing, or as a prescreening method for minimizing the amount of animal testing which may be required. These and other advantages of the present invention will be apparent from the discussion and description hereinbelow.

### BRIEF DESCRIPTION OF THE INVENTION

The present invention is directed to a toxicoproteomic method for preparing an assay for determining if damage has occurred to a specific organ system of an animal. The method includes a step of administering to an animal an agent which will cause damage to a specific organ of the animal so as to produce a protein which is modified as for example by oxidation, abnormal folding, or the like. In a further step, blood serum which includes the modified protein is collected from the animal, and the modified protein is separated from other proteins which are present in the serum. In particular embodiments of the invention, albumins and other such high abundance, interfering proteins are removed from the serum prior to the isolation of the modified protein. Such purification may be carried out utilizing immunoaffinity resins and/or standard chromatography techniques such as size exclusion and ion exchange. Immobilized antibodies to oxidized amino acids or other damage-related modifications are then used to isolate damaged and oxidized, organ specific proteins from serum. In particular embodiments of the invention, the modified protein is identified by standard proteomic methodology. Separation of the modified protein may be carried out via electrophoresis or other techniques. In a particular embodiment, the step of determining if the presence of the modified protein is specific to the damage to the tissues of a particular organ comprises comparing the serum collected from the animal with serum collected from a like animal treated with a second agent which causes damage to a different organ system of the animal, and analyzing the serum from the second animal to determine if the modified protein from the first animal is also present therein. In addition, a literature search may be performed to determine if the protein is organ specific. If the protein is specific, an antibody to the protein is raised and used in a number of assay configurations, with and without antibodies to oxidized amino acid or other damage-related modifications, to yield quantitative and novel qualitative data on the protein.

### DETAILED DESCRIPTION OF THE INVENTION

The present invention is directed to a method for identifying biomarkers which are specific to tissue damage in particular organs. In a first step of the method, organ specific chemical damage is induced in an in vivo system, typically by the introduction of a particular chemical agent into the system. For example, in rats, carbon tetrachloride, CCl₄, is a known hepatotoxicant, N-(3,5-dichlorophenyl) succinimide (NDPS) is toxic to the kidney, and ozone is toxic to the lungs. The administration of toxicants in the in vivo system will result in the production of oxidatively modified tissue specific proteins. Typically, these are oxidized proteins; and it is to be understood that in the context of this disclosure, “oxidation” is used in its generic sense to indicate a chemical reaction in which the protein loses an electron. Oxidation may comprise coupling of oxygen, halogens or other groups to the protein. In some instances, oxidation is the result of a direct reaction between the toxicant and the protein; while in other instances, oxidation of the protein is a secondary effect resultant from inflammation induced by the toxicant. There are a number of amino acids on the surface of proteins which may be oxidized or otherwise modified. Some particular oxidized amino acids which will constitute biomarkers will include: nitrotyrosine, chlorotyrosine, and methionine sulfoxide. All of these oxidized amino acids have been identified in various damaged, diseased and otherwise stressed tissues. In other instances, the protein may be modified by processes which result in abnormal folding, cross-linking, or other such topological changes. It is to be understood that the modified proteins employed as biomarkers in the present invention may include intact proteins as well as protein fragments, polypeptides and the like; and all of such species are included within the general definition of a modified protein. In accord with the present invention, proteomics based methodology is used to determine the identity of any of such modified protein.

In a second step of the invention, blood serum is drawn from the animal at various time points after organ damage and subjected to partial purification using such techniques as immunoaffinity purification and standard chromatography methods to remove high abundance and other unwanted and/or interfering serum proteins. Blood serum typically includes a very large number of circulating proteins, most of which are not organ specific, and it is advantageous to remove at least a portion of these nonspecific proteins prior to undertaking any attempt to isolate modified proteins from the serum. Such initial cleanup will generally remove albumins, immunoglobulins, and other high abundance serum protein. One method for such sample preparation is disclosed in United States Patent Application 2002/0127739 A1 which was published on Sep. 12, 2002.

Following by the partial purification of serum specimens described above, the isolation of organ specific oxidized proteins in serum is accomplished by immunoaffinity purification using antibodies to any number of oxidized or otherwise modified amino acids such as nitrotyrosine, chliorotyrosine and methionine sulfoxide. This novel approach obviates the need for antibodies directed against organ specific proteins at this isolation step yet it purifies proteins very likely to be tissue specific since oxidative changes occur predominantly in the target organ and oxidized protein present in serum after organ damage are likely to be derived from the damaged organ. The isolated proteins are then subjected to standard proteomics techniques, i.e., both one-dimensional and two-dimensional SDS-PAGE (sodium dodecyl sulfate-polyacrylamide gel electrophoresis) followed by such techniques as matrix-assisted laser desorption-ionization mass spectrometry (MALDI MS) or LC/MS to identify the proteins.

Once a modified protein(s) has been identified, the suitability of such protein as a biomarker must be validated by determining if the presence of the modified protein in the serum of the animal is related to damage to in a specific organ. This is necessary to avoid nonspecific or multi-specific biomarkers which may be indicative of tissue damage in other organs. Such validation may be accomplished by parallel testing of a number of agents having toxicity against different organ systems, in a particular animal model. In this approach, modified proteins are isolated from each of the test series, and those modified proteins which appear in tests involving different organ systems are deemed to be nonspecific and rejected. A second method to determine organ specificity is to perform a literature and protein database search.

Once a particular modified protein which is specific to tissue damage in a particular organ is identified, this protein can provide the basis for a highly specific assay system. Toward this end, antibodies are raised to this biomarker, and these antibodies in addition to antibodies against oxidized amino acids or other amino acid modifications, may be employed in various types of immunoassays to determine both the concentration as well as the type and extent of the protein modification.

It will be appreciated that through the use of the present invention, a series of biomarker proteins indicative of toxic damage to specific organ systems may be developed. Antibodies may be raised to this series of proteins, and these antibodies can be disposed in an appropriate assay format to allow for rapid and effective screening of compounds. For example, a compound under investigation may be administered to an animal, and serum collected from that animal. The serum can then be rapidly scanned utilizing a microarray analysis to determine which, if any, organ systems of the animal are adversely affected by the compound. As will be appreciated, the test can also provide for quantification of the severity of tissue damage as well as the determination of the type and extent of the modification of the protein. This data will provide novel information on the nature of the tissue reaction to toxicants and disease pathogenesis and development.

In drug development studies and other pharmacological applications, newly developed drugs may be rapidly screened for toxicological effects. Compounds manifesting significant toxicities can then be rejected. This approach is in keeping with the “fail fast and cheap” paradigm of new drug evaluation. Conversely, compounds having no toxicity or low toxicity may be further evaluated in conventional wide scale animal tests.

The assays produced in accord with the present invention may also be employed for health screening purposes. For example, a population may be screened for disease conditions utilizing the assays of the present invention. It is known that toxic exposures and particular disease conditions such as cancer, infections, autoimmune reactions and the like can produce oxidative stress in tissues which can result in the production of oxidized or otherwise modified proteins. Accordingly, a population may be economically and rapidly monitored utilizing assays of the present invention to look for tissue specific modified proteins. Those proteins may be indicative of toxic insult or disease development, and persons testing positive for such biomarker protein can be further evaluated utilizing more expensive and/or invasive techniques.

Through the use of the present invention, a panel of biomarker based assays can be created. These assays will provide toxicology data relating to any number of specific organs, as well as data relating to the specific biochemical pathways of toxicity within specific organs. The availability of sensitive biomarkers of toxicity will, under certain circumstances, eliminate the need for high dose to low dose linear extrapolation in toxicological testing. The present invention will provide for a reduction in the number of animals required for testing per agent, and will allow exploration of toxicity at an earlier stage in drug development thereby further decreasing time and expense.

Antibodies to the modified proteins developed through the techniques of the present invention may also be employed to determine the type (qualitative) and extent (quantitative) of modification of individual, organ-specific proteins. Such information will provide valuable and unique data regarding organ-specific toxicology and can be employed to develop biomarkers of peripheral (blood) oxidant/antioxidant status. Data developed using the methods and materials of this invention will include information such as whether increasing doses of the toxicant causes an increase in the concentration of the biomarker, or an increase in the oxidation of the individual amino acids comprising the protein, or both. Also, simultaneous measurement of several biomarkers for a given organ may provide a “signature” of specific damage that may provide additional diagnostic information.

### Experimental

A series of experiments were carried out to demonstrate and validate the various steps of the present invention. Lung damage was produced in rats breathing ozone at 0.8 and 2.0 ppm. Experimental groups consisted of three experimental and two control animals at each dose and specimens were collected at zero, one, two, four, eight, sixteen and twenty-four hours following ozone administration. In another experimental series, CCl₄ was administered at a level of 0.1 and 1.0 mL/kg to rats to induce liver damage, and specimens were collected at time points as described above. In both series of experiments, high abundance proteins in serum were found to contribute to nonspecific binding in subsequent steps; furthermore, it was found that their presence prevents one from loading a sufficient amount of oxidized proteins onto an electrophoresis gel. Therefore, these high abundance proteins are preferably eliminated from serum samples prior to further processing.

In this experimental series, the specimens were purified by passing them over affinity resins containing antibodies against whole rat serum proteins and collecting the eluate. This purification methodology was effective in removing a significant amount of the high abundant serum proteins, which typically have a molecular weight in the range of 49-80 kiloDalton. The bulk of these undesirable serum proteins have been found to have a relatively high and narrow molecular weight range, and oxidized serum biomarkers have been found to have a relatively low molecular weight. Therefore, it was determined that concentration of the sample could be carried out very effectively by a process of molecular weight sieving which retained low molecular weight proteins (typically 50 kiloDalton and below) and excluded high molecular weight proteins. Such sieving was effectively accomplished utilizing a Superdex 75 column with a sample loading volume of 10% of the bed volume. Processes of this type have the advantage of being cheaper and faster than affirity resin based processes.

Once the concentration of high abundance serum proteins was decreased, the tissue proteins were purified from serum by immunoaffinity resins using antibodies to nitrotyrosine, chlorothyosine and methionine sulfoxide. The isolated proteins were then separated by 2D gel electrophoresis which revealed a number of modified proteins found in the experimental group not found in the control group. Samples of interest were excised from the gel and those with sufficient concentration were analyzed by LC/MS.

It was found that three out of the seven peptides from the CCl₄ group were associated with mitochondria. They were glycerol 3-phosphate dehydrogenase, acetyl-CoA-acyl transferase, and electron transfer flavoprotein, alpha subunit. In addition to being restricted to cytosol, these proteins are associated with the organelle that is a source of superoxide, a major site of peroxynitrite formation when NO is present, and a major player in apoptosis. The fourth CCl₄ related protein that was identified is peroxiredoxin, the acidic variant of which is useful as a marker of oxidative damage to cells.

In the serum of ozone treated animals, four proteins of interest were found. Three are cellular proteins associated with plasma membranes: G protein-coupled receptor kinase; SPA-1, a Rap 1-specific GTPase-activating protein; and a potassium channel protein. The latter protein is activated by mitochondria derived ROS. A fourth protein was identified as nucleo-related protein usually associated with periribosomal particles. The CCl₄-induced and ozone-induced proteins differ and are specific biomarkers for toxin-induced liver and lung damage respectively. As is evident from the foregoing experiment, the methodology of the present invention is effective in producing, separating and identifying modified proteins which are indicative of toxic damage to particular organ systems. These experiments showed that without exception, all of the proteins examined in serumn were derived from the cytosol and are not members of the serum protein group. Thus, proof of the concept for the use of antibodies to modified amino acids for the isolation of tissue-specific, damaged proteins found in serum has been clearly demonstrated.

As will be apparent from the foregoing, the present invention may be readily extended and adapted to a variety of applications. The foregoing discussion, description and examples are indicative of particular embodiments of the invention, but they are not meant to be limitations upon the practice thereof. It is the following claims, including all equivalents, which define the scope of the invention.

1. A toxicoproteomic method for preparing an immunoassay for determining if damage has occurred in a specific organ of an animal, said method comprising:
administering to an animal an agent which will cause damage to a tissue of an organ of said animal, said damage resulting in the production of a modified protein, which modified protein consists of a protein which includes an oxidized amino acid and/or manifests an abnormal topology, said modified protein not being an adduct of a protein and said damage-causing agent, said modified protein being associated with or linked to the damage of said tissue or organ;
collecting blood serum from said animal, said serum including said modified protein;
separating said modified protein from other proteins which may be present in said serum;
determining if the presence of the modified protein in the serum of said animal is specific to the damage to the tissues of said organ, and if the presence of said modified protein is specific to said damage, then;
raising an antibody to said modified protein; and
employing said antibody to develop an immunoassay.

2. The method of claim 1, wherein said antibody to said modified protein is an antibody to a modified amino acid.

3. The method of claim 3, wherein said immunoassay detects the type of modification manifested by said modified protein.

4. The method of claim 3, wherein said immunoassay detects the extent of modification manifested by said modified protein.

5. The method of claim 1, including the further step of identifying said modified protein.

6. The method of claim 1, wherein the step of separating said modified protein from said other proteins is carried out via electrophoresis.

7. The method of claim 7, wherein said electrophoresis comprises two-dimensional electrophoresis.

8. The method of claim 7, wherein high abundance serum proteins are removed from said serum prior to said step of electrophoresis.

9. The method of claim 9, wherein said high abundance serum proteins include albumin.

10. The method of claim 1, wherein the step of determining if the presence of said modified protein is specific to the damage to the tissues of said organ comprises comparing the serum collected from said animal with serum collected from a like animal treated with a second agent which causes damage to a different organ system of said animal, and analyzing said serum from said second animal to determine if said modified protein is present therein.

11. The method of claim 1, wherein the step of determining if the presence of said modified protein is specific to the damage to tie tissues of said organ includes the step of identifying the protein which was modified to produce said modified protein and consulting a database to determine if said protein is specific to said organ.

12. The method of claim 1, wherein said organ system is selected from the group consisting of: lungs, liver, and kidney.

13. A toxicoproteomic method for identifying a biomarkcer which is indicative of damage to a specific organ system of an animal, said method comprising:
administering to an animal an agent which will cause damage to a tissue of an organ of said animal, said damage resulting in the production of a modified protein, which modified protein consists of a protein which includes an oxidized amino acid and/or manifests an abnormal topology, said modified protein not being an adduct of a protein and said damage-causing agent, said modified protein being associated with or linked to the damage of said tissue or organ;
collecting blood serum from said animal, said serum including said modified protein;
separating said modified protein from other proteins which may be present in said serum; and
determining if the presence of the modified protein in the serum of said animal is specific to the damage to the tissues of said organ, wherein if the presence of said modified protein is specific to said damage, said modified protein comprises a biamarker indicative of damage to said organ.

14. An immunoassay for detecting damage to a specific organ of an animal, said immunoassay comprising:
an antibody to a modified protein which is characteristic of damage to said organ, said antibody being produced by a process which comprises:
administering to an animal an agent which will cause damage to a tissue of said specific organ, said damage resulting in the production of a modified protein, which modified protein consists of a protein which includes an oxidized amino acid and/or manifests an abnormal topology, said modified protein not being an adduct of a protein and said damage-causing agent, said modified protein being associated with or linked to the damage of said tissue or organ;
collecting blood serum from said animal, said serum including said modified protein;
separating said modified protein from other proteins which may be present in said serum;
determining if the presence of the modified protein in the serum of said animal is specific to the damage to the tissues of said organ, and if the presence of said modified protein is specific to said damage; and then,
raising an antibody to said modified protein.
